(19) United States
(12) Patent Application Publication
(10) Pub. No.: US 2026/0123998 A1
(43) Pub. Date: May 7, 2026

(54) IMAGE-BASED SURGICAL GUIDANCE

(71) Applicant: Vent Creativity Corporation, New York, NY (US)

(72) Inventor: Gokce Yildirim, Weehawken, NJ (US)

(73) Assignee: Vent Creativity Corporation, New York, NY (US)

(21) Appl. No.: 19/440,458

(22) Filed: Jan. 5, 2026

Related U.S. Application Data
(63) Continuation of application No. PCT/US2025/047532, filed on Sep. 23, 2025.
(60) Provisional application No. 63/699,456, filed on Sep. 26, 2024.

(51) Int. Cl. A61B 34/10; A61B 90/00

(52) U.S. Cl. CPC A61B 34/10; A61B 90/361; A61B 2034/105; A61B 2034/107; A61B 2090/365

(57) ABSTRACT

A process includes graphically guiding a surgical workflow of a surgical procedure. The graphically guiding includes tracking object(s) by obtaining point cloud data based on imaging field(s) of view during the surgical procedure, and applying an artificial intelligence model to the point cloud data, and recognizing (i) object(s) in the field(s) of view and (ii) positioning of the object(s) in the field(s) of view. The object(s) include an object to be placed as part of the surgical procedure. The process displays graphical element(s) corresponding to the object on a display device and in a desired position relative to patient anatomy displayed as a live view to the patient anatomy and/or as a digital model of the patient anatomy on the display device, and updates properties of the graphical element(s), where the updating is based on detected positioning of the object in the field(s) of view, as informed by the tracking.

### CROSS-REFERENCE TO RELATED APPLICATIONS

This application is a continuation under 35 U.S.C. 111 (a) of International Application Number PCT/US2025/047532, entitled “IMAGE-BASED SURGICAL GUIDANCE”, filed Sep. 23, 2025, the entire contents of which are hereby incorporated by reference herein, and claims the priority benefit of U.S. Provisional Application No. 63/699,456, filed Sep. 26, 2024.

### BACKGROUND

Surgical plans and surgical cutting guides are often used by surgeons to place (for instance: position, move, navigate, etc.) implants, instruments, and other three-dimensional (3D) objects in a surgical site for various purposes. The lack of accuracy and subjectivity of this process is well documented. Whether in a manual surgery or a navigated setting, surgeons place instruments based largely on experience or other subjective considerations. In any case, the placement of objects facilitates actions related to the surgery, for instance to execute cuts and place appropriate implants to improve patient conditions.

The placement of these objects is based on various plan options. However, in some cases, these are led primarily by relatively simplistic visual guides like two-dimensional (2D) x-ray images. Using the example of a knee surgery, a single coronal plane x-ray image, or a combination of coronal plane x-ray image and sagittal plane x-ray image, might be relied upon by a surgeon to assess generally where the implant is to be positioned. Often a calibrated image of the implant, such as a transparent sheet with an outline of the implant, is placed over the x-ray(s) to determine an approximate desired implant location. However, there are inherent problems when positioning a 3D object based on a 2D representation of the anatomy. The limited information provided by the simplistic visual guide coupled with limited or no ability to exactly recreate the intended surgical approach can be problematic to delivery of the desired surgical outcome. Even in robotic surgical scenarios in which precise robotic cutting is employed, the robots are not used to place implants or other objects. As these tasks remain for the surgeon and/or other medical practitioners to perform, they remain susceptible to error.

### SUMMARY

Shortcomings of the prior art are overcome and additional advantages are provided. In one or more embodiments, a computer-implemented method is provided that includes graphically guiding a surgical workflow of a surgical procedure. The graphically guiding includes tracking one or more objects by: obtaining point cloud data based on imaging one or more fields of view during the surgical procedure; and applying an artificial intelligence model to the obtained point cloud data, and recognizing, based on the applying, one or more objects in the one or more fields of view, and positioning of the one or more objects in the one or more fields of view, the one or more objects including an object to be placed as part of the surgical procedure. The graphically guiding further includes displaying at least one graphical element corresponding to the object on a display device and in a desired position relative to patient anatomy that is displayed as a live view to the patient anatomy and/or as a digital model of the patient anatomy on the display device, and updating properties of the at least one graphical element, wherein the updating is based on detected positioning of the object in the one or more fields of view, as informed by the tracking.

Additionally or alternatively, in one or more embodiments the object includes an implant to be placed relative to the patient anatomy. Additionally or alternatively, in one or more embodiments, the object includes a surgical alignment guide. In one or more embodiments the surgical alignment guide is a cut guide, the at least one graphical element includes at least one representation of the cut guide, and the method further includes displaying, on the display device, additional graphical elements that correspond to cut planes associated with cuts be made. Additionally or alternatively, in one or more embodiments the object includes a surgical tool used in the surgical procedure.

Additionally or alternatively, in one or more embodiments, the method further includes displaying, on the display device, an additional graphical element that corresponds to a drill hole location or screw placement location, where the displaying the additional graphical element includes positioning the additional graphical element in a desired position for the drill hole or screen placement relative to the patient anatomy that is displayed as a live view and/or as a digital model on the display device.

Additionally or alternatively, in one or more embodiments, the display device is a display device of smart glasses through which a user has a field of view of the one or more fields of view.

Additionally or alternatively, in one or more embodiments, the properties of the at least one graphical element include one or more colors or shading of the at least one graphical element.

Additionally or alternatively, in one or more embodiments, the patient anatomy includes bone and/or soft tissue having varying densities or tension at different portions of the patient anatomy, and the method further includes displaying an additional graphical element representing the patient anatomy, where different portions of the additional graphical element correspond to the different portions of the patient anatomy, and wherein displaying the additional graphical element varies properties of the additional graphical element at the different portions of the additional graphical element according to the varying densities or tension at the corresponding different portions of the patient anatomy.

Additionally or alternatively, in one or more embodiments, the method includes training the AI model by an object calibration approach that includes: pre-operatively imaging the object to be placed from varying angles and capturing a point-cloud of the object to be placed; and training the AI model using the point cloud to recognize the object to be placed during the surgical procedure.

In accordance with one or more aspects, each of the embodiments is separable and optional from one another. Further, embodiments may be combined with one another.

Computer-implemented methods, computer systems, and computer program products relating to one or more aspects may be described and claimed herein. For instance, a computer system can include a memory and a processor in communication with the memory, where the computer system is configured to perform any of the above or herein-recited method(s)/process(es). As another example, a computer program product can include a computer readable storage medium readable by a processing circuit and storing instructions for execution by the processing circuit to perform any of the above or herein-recited method(s)/process(es). Each of the embodiments of a system, method, or computer program product may be embodiments of each system, method, and/or computer program product. Further, each of the embodiments is separable and optional from one another. Moreover, embodiments may be combined with one another.

The present summary is not intended to illustrate each aspect of, every implementation of, and/or every embodiment of the present disclosure. Additional features and advantages are realized through the concepts described herein.

### BRIEF DESCRIPTION OF THE DRAWINGS

FIG. 1 depicts an example conceptual workflow of aspects described herein;

FIGS. 2A-2F depict examples of graphical elements displayed to facilitate a surgical procedure, in accordance with aspects described herein;

FIG. 3 depicts an example process for graphically guiding a surgical workflow of a surgical procedure, in accordance with aspects described herein;

FIG. 4 depicts one example of a computer system and associated devices to incorporate and/or use aspects described herein; and

FIG. 5 depicts one example of a smart glasses device.

### DETAILED DESCRIPTION

Described herein are tracking and targeting solutions for surgical applications. The solutions can be easily followed for high-accuracy and objective placement of implants, instruments, and other objects, for instance.

An advanced approach employs a robotic and navigation-based system, in which tracking guides are placed onto secondary sites on patient bones. Tracking guides are tracked by the navigation system, and a user is to generate landmarks on the bone surfaces to inform the system where each bone is. However, this process can be a time-consuming workflow, and tracking guides introduce additional infection and fracture risks. Further, after cuts are made, there is no indication to show a precise location for placement of the implant to achieve the preoperative plan, and no confirmation that the implant has been properly placed.

In accordance with aspects described herein, any of various surgical tracking technologies may be used for guiding surgical actions such as, but not limited to, cutting and object positioning, including implant positioning. Example technology used for tracking is any wave-based imaging approach, such as Light Detection and Ranging (LiDAR), or more generally any ‘time of flight’ procedure that can provide spatial information in the form of point coordinates of object surfaces in 3D space. Approaches leveraging LiDAR, infrared (IR) light, microwaves, or radio-waves are just some examples. In some aspects, a system (e.g., a computer system, which could be any of various types, examples of which include a cloud computing system, local or remote server, main frame system, desktop computer, workstation, mobile device, and/or smart device, such as smart glasses, an example of which is augmented reality (AR) glasses) can track position, movement, orientation, and other spatial characteristics of objects within a surgical site. Example objects include patient anatomy (tissue, bone, etc.), surgical instruments, and equipment in a surgical suite, though any other type of object desired could be tracked.

In embodiments, the tracking technology is leveraged to create a point cloud survey of at least some of the field of view of an imaging device (or imaging devices that collectively provide image data to image the field of view). An imaging device may be interchangeably referred to herein as ‘camera’, and the imaging device(s) and any associated hardware/software may be referred to as a ‘vision system’ herein for ease of reference. In some examples, the camera is provided as part of a smart device, such as smart glasses that incorporate camera(s) and, in general, image a field of view of a user wearing the smart glasses. Imaging a field of view provides point cloud(s). These point clouds, if presented to users graphically on a display (which could include a display provided as part of the smart glasses, or a display associated with another computer system), can have at least some points distinguished from other points using one or more visual characteristics, such as a varied color characteristic, based on surface depth. As an example, the deeper into the field of view a point sits (such as a crater on the surface of bony anatomy shown in the field of view), the darker the color that is used to color that point when the point cloud is rendered for user view. In an embodiment in which a user wears AR glasses, the user could perform a gesture that is recognized though imaging or other input to the AR glasses and informs a region of interest in the user's field of view, which region can then be tracked and specifically compared to other regions, and potentially the rest of the field of view of the user. Further with respect to bone anatomy, in some embodiments density-based point clouds are provided, in which different colors are used for the points corresponding to different bone densities at those points. Additionally, soft tissue objects, such as cartilage, meniscuses, ligaments, and muscles, can be segmented and animated from the ground truth image to display for the surgeon intraoperatively through a screen/display/monitor and/or AR glasses. This can be helpful because when the point cloud/bone model is displayed showing such color variation along with a graphical element representing an implant, the surgeon can easily see bone density relative to implant positioning and help ensure that the implant is positioned in a desired position given the bone density reflected. Additionally, locations of high bone density are known to indicate ligament attachment (tension) and bone contact (compression). This can be used to model the posture of the patient, and therefore reflect how the patient's joint is likely, or most frequently, to be postured. Further, ligament attachments can be connected from one relatively-dense patch to another relatively-dense patch with individual lines to measure and minimize tensions to prevent strain when planning implant or soft tissue repair surgeries. The resulting ligaments can be displayed as anatomical features in real time, and also can show color changes corresponding to slack or tensioned states to help guide the surgeon on the optimal joint line as the surgeon moves the joint in passive and/or active loading scenarios.

In any case, the point clouds can be built and/or obtained by a system (such as a system noted above). The system can have a trained artificial intelligence (AI) model that can detect and identify various objects based on point cloud(s) that are provided as input to the model, and in order to facilitate real-time tracking of those objects. Any of various AI model(s) may be appropriate for this task. In particular examples, the AI model is a trained deep neural network (DNN).

In some examples, the AI model is trained based on an object calibration approach in which an object that a user desires to be subsequently recognizable when placed in a field of view is pre-scanned to learn a point-cloud of the object. In the case of a surgical instrument, object calibration might involve a user placing the instrument on a surface and imaging the object from various angles using an imaging device, for instance one that is capable of sensing depth, such one using LiDAR-based imaging, or other imaging device, as noted above. Once the system acquires an adequate point cloud of the object, the AI model is trained to recognize that object. This recognition might be useful in situations where, subsequently, a point cloud of the object is captured and input to the AI model for identification. The object can thereby be recognized by applying the AI model to a point cloud of the object later-obtained when imaged in a field of view of the camera. Similarly, a cutting guide, implant, or any other object can initially be scanned with an imaging device from various points of view as part an object calibration procedure to learn the point cloud of the object and train the AI model on that point cloud. In particular examples, a Simultaneous localization and mapping (SLAM) procedure is undertaken to construct a model of the object, which is then provided as, or translated into, a point cloud for training the AI model.

It should be noted that ‘training’ the model on a given object may be sufficient to train the model to identify different objects that are sufficiently similar to the given object. For instance, training the model to recognize a given sagittal saw may be effective for the AI model to identify a different sagittal saw if a point cloud of that different sagittal saw were input to the AI model and were sufficiently similar to the point cloud on which the AI model was trained. As another example, training the AI model on one retractor may be sufficient for the AI model to recognize a different retractor having a sufficiently-similar physical profile if a point cloud thereof is input to the AI model.

It should be further noted that the AI model could be trained on computer models of objects, rather than images of the objects. For instance, a computer-aided design (CAD) model of an implant or any other object could provide the information needed (e.g., surface topology) to train the AI model to recognize the object based on an input point cloud of the object.

In any case, the AI model is trained to recognized objects, and the system can leverage the AI model to recognize the objects when in a field of view of imaging devices that provide image data from which object point clouds are constructed. Further, the system can track those objects in the field of view based on continual imaging of the field of view, generation of the point clouds of those objects, and recognition of the objects. Thus, the objects can be tracked in real time when seen in the operating room (OR) and during a surgical procedure. This may be useful to inform object positioning, as is described in additional detail below. For instance, using this technology, a 3D printed alignment guide can be placed on the patient (in the field of view), and the system can register that to the medical images from which the surgery was planned. In additional embodiments, the model can be trained to recognize the hands of a user (e.g., a surgeon) and specifically to recognize gestures and/or an areas to which the user points, which area can be identified as a region of interest for the system to track specifically for object registration procedures, such as bone instant registration without a need for 3D printed alignment guides, to provide registration of the bone for display of preplanned cuts, implant positions, areas of interest, etc.

In some embodiments, graphical element(s) are displayed on a display device (such as a monitor, television or similar, transparent display, or any other active or passive display) to show locations and positioning of objects or other features relevant to the surgical procedure being performed. For instance, augmented reality (AR) graphical elements and/or other graphical elements may be used to show locations of various critical elements of the surgical procedure. Graphical element(s) could be placed into images streamed on a display in the operating room and/or into a user's line of sight to the environment through a transparent display, such as a transparent display of smart glasses that the user wears, as examples. Smart glasses are also referred to as AR glasses herein and are sometimes also referred to as ‘AR goggles’.

As an example, graphical element(s) could be placed to show a user (surgeon or other medical practitioner) the position of cut plane(s) for cuts to be made. In some scenarios, the cut planes are placed relative to a recognized alignment guide. In other words, the alignment guide may be recognized in the field of view by way of the point cloud approach discussed herein, and the system, having knowledge of the intended cut plane positioning relative to the alignment guide, can interpose cut planes into the field of view of the user and/or on a display that also shows the alignment guide positioned on the patient. Since positioning of objects in the field of view can change based on object movement or movement of vision system components (e.g., the camera(s)), the imaging of the surgical site, recognition of the object(s) in the field of view, and display of the graphical elements could be continually performed so that even if objects in view, such as patient anatomy, instruments, and other objects, move relative to the vision system, the graphical elements remain correctly positioned relative to the other objects shown. Thus, the system can track the position of the alignment guide and update the location/position/orientation of the graphical elements corresponding to the intended cut planes on the display. More broadly, graphical elements could be placed for any of various other types of indications-drill holes or screw placements for sports medicine ligament repair or meniscus repair type surgical interventions, for instance. Many others are possible. As another example, bone density point clouds and ligament fibers derived from attachment sites can also be visualized/highlighted with graphical elements, optionally with color-coded and/or color-based representations of characteristics of those elements, for instance the percentage length of the individual fibers relative to their taught 100% lengths after the surgeon ‘shows’ that taught state to the system. That taught state could be shown in various ways, for instance by manipulating an anatomical model or manipulating the actual patient anatomy during the surgical procedure. Features such as the growth plate of a human joint can also be isolated to calculate joint lines independently of the arthritic joint line which changes with increased disease state.

In an AR embodiment in which multiple users wear smart glasses or otherwise see a live view within the operating room via imaging device(s), all such users can nevertheless see graphical elements and track recognized objects for various applications. It is noted that at any given time, different users might view different areas of the operating room, or might view the same, or a substantially overlapping area of the operating room (for instance surgical site of the anatomy), albeit with potentially varying lines of sight. In any case, the graphical elements provided in the view of different users might differ. For instance, after placement of the alignment guide and before cuts are to be made, the surgeon viewing the guide and anatomy might see graphical elements corresponding to the intended cut planes, while the nurse looking at an instrument table might see graphical elements that highlight a required next instrument or an implant to be placed. As described above, the AI model(s) responsible for object recognition can be prior-trained to recognize any objects desired, such as any objects within the OR for which recognition may be helpful. Then if the surgeon or another user triggers a step in the surgery, any or all users can be alerted to any relevant object(s) for that step, for instance a retractor or other object. Different users might utilize different instruments at a given step, and each user can be shown graphical element(s), for instance shown in AR via AR glasses that they wear, which relevant object(s) are needed when those objects are in the user's field of view. In particular examples, a user, such as a surgeon, can provide a spoken or other audible cue for a next instrument or other object needed. The instrument/object can be displayed/highlighted to the user and/or other user(s) to facilitate fast identification of objects desired, which results directly in increased OR efficiencies.

This concept can be applied to various steps of a surgical procedure. After cut(s) have been made, graphical element(s) can show proper placement of an implant on the patient anatomy so that the exact size and position of the implant can be seen by the surgeon, for instance via the surgeon's AR glasses or on a display that streams a view to the surgical site.

Graphical elements corresponding to patient anatomy could also be displayed. For instance, in the case of limited approach surgeries, such as a minimally invasive surgery, where some relevant anatomy may be unexposed, graphical models of the anatomy, such as a patient bone, could be displayed to overlay the imaged patient skin (i.e., to show at least portion of the unexposed bone) at the exact location thereof. In an example of sports medicine or soft-tissue surgeries, the system can track k-wires or similar small objects and guide the surgeon to the area of interest for intervention. In these examples, some axial information of objects may be provided, for instance through imaging an end portion of an object, and then used to track the object.

Similarly, the system can work with arthroscopy tools that include 2D cameras to stream images of internal anatomy. In this case, if the instrument geometry (length, etc.) is known, for instance through object calibration or otherwise, then this can be used in conjunction with magnetic resonance imaging (MRI) or other pre-operative imaging data to figure out from the provided 2D images where the instrument is in three dimensions relative to other objects. As another example, the system could track probes used for assessing soft tissue integrity, and leverage them as digitization tools in a 3D tracked environment to register the bone locations relative to the camera or AR goggles. In this embodiment, the system can then display the MRI or CT-based bone images with the tunnel or other intervention plans in 3D independent of the 2D image displayed in the OR in order for the surgeon to see through the display/AR glasses and operate using the spatial information provided. These examples can be used to ensure proper angling of the arthroscope to provide a correct tunnel trajectory, for instance.

As another example of tracking in accordance with aspects described herein, power tools used may be tracked and graphical elements relative to the actions performed with them can be displayed to facilitate accurate use of these tools. For instance, in the case of a drill, graphical elements showing point locations, axial alignment, or other characteristics can be displayed to help the user align the drill to an exact angle and point of entry.

Particular embodiments of a disclosed system include backend component(s) and front-end component(s), though there may be other components that sit between the front-end and backend in terms of data exchange and/or processing. Example backend component(s) can be systems, such as computer systems, which could reside anywhere, including in a cloud platform, or on-site at the surgical center or hospital, as examples. Example front-end systems could be computer systems in the OR, examples of which include regular workstations/desktops, or mobile devices such as laptops, smartphones, or smart glasses, such as AR glasses. The vision system-imaging devices and potentially computer system(s) performing data gathering/processing of the imaging data therefrom depending on the computing tasks handled by the imaging devices-could be considered front-end component(s), as they are provided in the OR. In examples, surgical planning is performed on one or more backend systems and software executing thereon. Surgical planning can include cut planning and implant placement, among many other planning.

In examples, maintenance of the AI model(s)—including building, training, verification, retraining, etc.—are handled by backend component(s), though they may alternatively be delivered by a third party to backend component(s). Object calibration to train on model(s) instruments could be handled by any systems/components. In examples, front-end components like smart glasses are used to image an object in the OR or otherwise, and then provide the image data to another system, such as a backend component or other device, to build a point cloud of at least a portion of the object. The AI model can be trained on this to recognize the object. This may be performed in real-time just before the surgery in which the object is to be used, or even during the surgery, in some embodiments. Alternatively, this could be done outside of the OR, by object manufacturers that provide image data, point cloud data, or other object models on which AI models can be trained, as examples. Many approaches are possible.

In a particular embodiment, the surgical plan (in the form of data) may be provided to/on an on-site system, for instance a system that is resident in the OR during the procedure. In examples, the plan is uploaded to smart glasses worn by one or more users in the OR. For instance, there could be a synchronization procedure that synchronizes the plan when the smart glasses are plugged into another system, such as a computer system, at the surgical facility. That computer system, which could have built the surgical plan or obtained the surgical plan from another system, such as a cloud-based surgical planning system, can synchronize the plan to the smart glasses when they are plugged into the system or synced wirelessly. In examples, the glasses are plugged in for charging and this prompts the synchronization of the plan to the glasses. Additionally or alternatively, the smart glasses may be in wired or wireless communication with another system during the surgical procedure in order to send/receive data during the procedure. Thus, in a particular embodiment, the surgery is planned by or using a cloud system and then it is ‘run’ offline during surgery directly on smart glasses and/or another system in the OR.

In accordance with some embodiments, a general workflow includes preoperative acquisition of image(s), such as computed tomography (CT) images of patient anatomy, and creation, based on that preoperative imaging, of anatomical model(s), such as point cloud(s) or other 3D model(s), of patient anatomy, including bone, for example. Such bone models can reflect density of depicted objects such as patient anatomy. The models could reflect bone density, including places where the bone is most and least dense, as well as ligament stiffness derived from the image density calculations to alert the surgeon to the areas of stiffness or concern.

The preoperative imaging can provide an anatomical model against which point cloud data can be referenced to provide a robust view of the surgical site and facilitate proper placement of graphical elements, in augmented reality or otherwise, display during surgery. In surgery, a point cloud or mesh representation, as examples, of the patient anatomy, e.g., bone, ligaments, cartilage, meniscus, arteries, etc., may be displayed. This can be displayed on a display device that displays streams of images of the anatomy during surgery, or displayed on a display device that is in a line-of-sight of the user, for instance on a transparent display through which a user views the surgical environment. In any case, graphical elements showing planned cut planes (as an example) can be displayed. These graphical elements could be colored planes, as an example.

As the surgeon views the patient anatomy, one objective may be to identify location and pose of anatomy, and therefore a registration of anatomy is performed. As examples, (i) a tracking guide in the form of dedicated tracker(s) or those provided as part of an alignment guide or other object, or (ii) a scan of the anatomical (e.g., bone) surface is used to register the patient anatomy in real space to the patient anatomy model previously created, though other ways of registration, such as the use of registration gloves discussed below, could be used. The registration may be used to help determine the location of surgical actions, such as cuts. For instance, registration could be used to determine where, in real space, cut planes are to be positioned, i.e., where they sit within in the live view to the surgical site. In this regard, there are at least two potential modes-one shows a desired cut plane (e.g., in AR) and the surgeon can free-hand execute the cut using the cutting tool to execute the cut along the cut plane, and the other shows (e.g., in AR) where a cutting guide is to be placed such that a cutting guide slot of the cutting guide aligns with the desired cut plane. The latter may be more appropriate in instances of manual surgery where the surgeon prefers not to free-hand cut but desires to use a manual instrument instead. Relative to manual instruments placed using conventional heuristics, aspects described herein show the exact location to place the instrument, thereby resulting in increased accuracy. Thus, the cut planes and/or guides placed based on cut plane positioning can be graphically represented on the display for the surgeon, and the surgeon can accurately execute the cuts. As a cut is made and/or completed, the corresponding graphical element(s) for the cut plane could be removed. Then once the cuts have been made, a graphical element could be displayed (e.g., as augmented reality for example) showing the exact positioning/location/orientation of the implant. In this regard, the implant characteristics including what the implant looks like, and its planned positioning is already known, as this was determined as part of preoperative or intraoperative surgical planning. This element can be displayed in the view at the proper location for the implant.

In accordance with some aspects, as the surgeon then brings the implant into the view, the system can recognize the implant based on its point cloud and begin to track it. As the surgeon places the implant, the graphical element showing the correct positioning of implant can be updated. That is, properties of the graphical element can be updated. In an example, the graphical element is initially one color, then as the implant is moved closer into position, this changes gradually or others to another color. In an example, the element starts out a shade of red that gradually changes to a shade of green as the implant gradually moves into proper position. Additionally or alternatively, the element can be displayed with coloring corresponding to a heatmap that updates/changes color depending on whether the implant is properly positioned in that area. For instance, the element might initially be a slightly opaque red as the implant is moved into the field of view, then gradually transition to an opaque green that becomes less opaque as the implant fills the volume of the graphical element. If the implant is mostly in position but is, say, rotated just slightly, the element could be mostly opaque green but slightly red in areas where the implant is not properly located. Once the implant is fully properly positioned, the element could turn bold green, as an example. In this manner, a spectrum or heatmap of any number of colors may be used to show where the implant and plan differ, in order for surgeon to potentially act on. Additionally or alternatively, the surgeon may be able to use a tracked registration glove as discussed below to ‘brush’ on the surface to verify the correct implant position based on the tracked glove.

In examples, the above may be provided in AR, though it could additionally or alternatively be provided on a standard display/screen showing an anatomy model of the patient and digital representation of the implant and its positioning. The user could look at a screen showing the digital representation of the implant (as the graphical representation displayed) and the anatomy model, and observe the change in that graphical representation as the user positions the implant on the patient anatomy. The properties (e.g. coloring) of the graphical element, as the implant model, can therefore update to reflect whether and when the implant, which is being tracked as described herein, is properly placed. The action to vary properties of a graphical element representing an object can be done for other objects as well.

In some aspects, this can also be done when presenting graphical elements corresponding to the recognized point clouds of objects. For instance, a graphical element can be imposed on the implant in the field of view and track over the implant as it is moved. The graphical element can change color as the implant is moved closer to its correct position. In the case of a drill object, a graphical element overlaying the drill bit could be updated (to change from red to green, for example) as the drill bit is moved closer to its intended point of anatomical entry. This could similarly be done for a saw blade making a planar cut. Additionally or alternatively, graphical element(s) could be provided showing intended locations of drill bit points of entry or saw blade points of engagement with patient anatomy. The system can additionally or alternatively reflect, via graphical elements on a display, and in some examples via AR elements in the field of view of user(s), the bone density of bone involved in surgical actions such as cuts or drilling. For instance, graphical elements could be provided to reflect bone density of bone material through which the cut plane passes or bone material in the cylindrical drill path of a drill bit. This reflection of bone density can help the surgeon assess the appropriateness of the surgical actions.

As noted above, the particular graphical elements displayed on, e.g., smart glasses or other displays, at any given time can be dependent on where the current process is in the surgical procedure. The system can, step-by-step and as the procedure proceeds, show appropriate graphical elements and coloring (or other properties thereof), thereof to keep users' attention focused on the tasks at hand. Coloring and other properties can be used to highlight tools appropriate for the current task, like retractors for performing a cut after an incision. Graphical elements can also show optimal locations for the tools used during a given task and can use color (or other property) changing to guide the users to those optimal location positioning. For example, a graphical element in the shape of the retractor could be placed at the optimal location for the retractor and could change color from red to green once that retractor has been properly placed.

In some embodiments, a user could trigger the system to provide graphical element(s) on display(s) for one or more users. For instance, sensors such as microphones could be used to detect verbal cues from a user. An example verbal cue could be the surgeon calling out for a mallet, which could trigger the system to recognize the mallet in the field of view of a nurse or other user and highlight that tool by imposing a graphical element on the display that the user views, for instance the AR glasses that the nurse wears or a display device that the nurse views. The nurse could thus turn to the instrument table that holds the mallet and immediately see (by way of the graphical element) where the mallet is. This could be particularly useful in situations where there are multiple, similar looking objects (like varying size implants) between which the system can easily distinguish because of their different point clouds, but that humans might not as easily distinguish from each other.

As noted previously, medical images are usually generated when a patient has been scheduled for surgery to support the case. Even without medical images, surgery can be scheduled for exploratory or other cases.

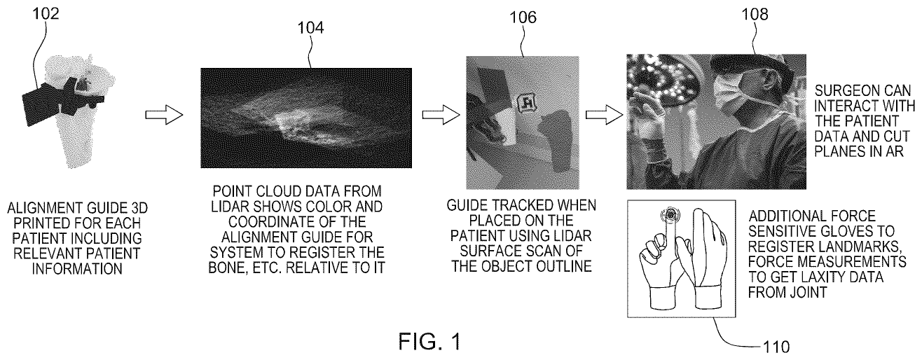

In any event, and referring to FIG. 1 as an example conceptual workflow of an embodiment, an alignment guide 102 can be 3D printed by the manufacturer and have specific features to identify it as an inorganic object. This can facilitate tracking of the object, in some examples. In accordance with aspects described herein, this object can be scanned with, e.g., a LIDAR scanner as a final step of manufacturing to generate point cloud(s) from the surface and train an AI network/model for identification and tracking of the guide in 3D space. Further, the alignment guide 102 can be associated with the patient information. This may be useful when recognized by the trained model when brought into a field of view during the planned surgery. Patient information as well as vital surgical information (such as the associated anatomy-left leg, right hip, etc.) can optionally be displayed in AR or otherwise. An alternative to a patient-specific alignment guide is a generic guide, which can be used for varying patients and can be tracked in accordance with the approaches discussed herein.

A LIDAR and/or IR scan may be taken of the alignment guide. The scan could show color and coordinates of the alignment guide for the system to register the bone relative to the guide. Continuing with FIG. 1, shown is an example point cloud 104 of the alignment guide 102 in 3D space. The AI model can be trained to recognize an input point cloud as corresponding to this guide. This enables the guide to be tracked (106) when placed on the patient. The tracking could be done using, e.g., a LiDAR surface scan of the outline of the object in the field of view to obtain a point cloud of the object, and then feeding that point cloud as input to the trained AI model. The trained model can then recognize the object as the implant, having been trained to recognize the implant.

In embodiments, during the day of the surgery the alignment guide is cleaned appropriately. After initial incision(s), the alignment guide is placed in accordance with what is reflected for the surgeon preoperatively, for instance on a reproducible location identified by the guide manufacturer. If the guide is not patient specific, force gloves or other gloves subject to tracking, for instance tracking by AR goggles, can be used to paint the relative area with the fingers of the surgeon, and the tracked hand from LIDAR/IR imaging can be used for an imageless option. That is, in order to register anatomy in real space, and instead of using a standard probe-based registration, a user could use gloves with sensors, for instance force sensors to register landmarks. The gloves could be tracked and/or have trackable elements to inform positioning in real space. When the user engages with contours of the patient anatomy, this informs the system of anatomical surface properties. In some examples, the gloves have force sensors that can be activated by the user (by pressing against the anatomy, performing gestures or providing audio prompts, for instance) to trigger data capture/send to a system.

If imaging was used, then when the alignment guide is placed, the cut plane positioning can be indicated by graphical elements as explained elsewhere herein. For instance, in some examples the alignment guide registers the bone, and then elements of the surgical plan, including cut planes, can be shown relative to the anatomy and/or other objects. In examples, the intended cut planes may be reflected on the display by graphical elements relative to the alignment guide reflected on the display (or in the live environment viewed through the display in the case of a transparent display). The surgeon may have the freedom to adjust the surgical plan. Some alignment guides may not force a single cut option, in which case the surgeon may be free to change the cut plan and be presented with the new cut plane. In examples, the surgeon interacts (108) with patient data to change cut plane angle or other properties. In other situations, preoperative imaging is not available. Anatomical landmarks can be requested/obtained in other manners, for example by force sensitive gloves, joint laxity data can be gathers from force measurements (110), and cut planes can be generated from those landmarks, alone, for display as graphical elements.

Once the cut planes are selected, the retractors, saws, drills, and/or any other objects on which the AI model(s) is/are trained and that appear in the field of view of any one or more users can be tracked with the system (e.g., leveraging a LiDAR-based and/or IR-based vision system for example) to show the surgeon and/or other user(s) where those objects are relative to the patient anatomy being operated on, which may also be tracked. As noted above, preplanned locations of these tools can be visualized in graphical elements, for instance in AR, if desired, and the surgeon and/or other staff can see the graphical elements on display(s) in the OR. Examples include AR glasses, as described. In these situations, users can adjust the positions of tools using to place them at planned, accurate locations.

After cut(s) have been executed such that the anatomy is ready to accommodate an implant, the implant can be introduced to the area. An implant in the field of view can be tracked based on the trained AI network/model as described, and graphical element(s) can optionally be displayed relative to that tracked object, if desired. Additionally or alternatively, proper placement of the implant—whether preplanned or updated at any point—can be visualized, and the proper location for the implant can be displayed via graphical elements, for instance in AR. This conceptually provides a ‘paint by numbers’ approach that shows the surgeon a clear indication of when the implant is expected to be when in the exact location. In examples, this is done by way of a changing visual characteristic to indicate (i) progression toward proper placement and/or (ii) that the implant has been properly placed. An example is changing a color, opacity, texture, or other visual property of the element. Other options for visual guidance to help with the ease of execution of the surgery may be provided.

By way of specific example, the color may be changed (for instance from red to green) once the implant has been properly placed on the anatomy. The user can move the implant around until it aligns with the corresponding graphical element(s) being displayed. Those elements can change from red to green (as examples) once that happens, or could transition from red to green (as examples) as the implant is brought closer to the indicated position. Other options are possible.

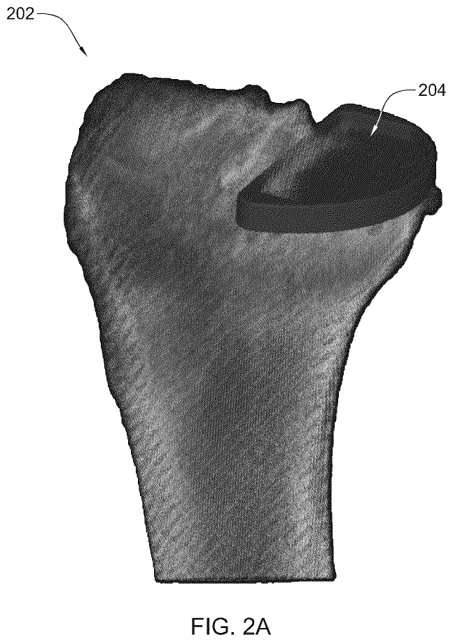

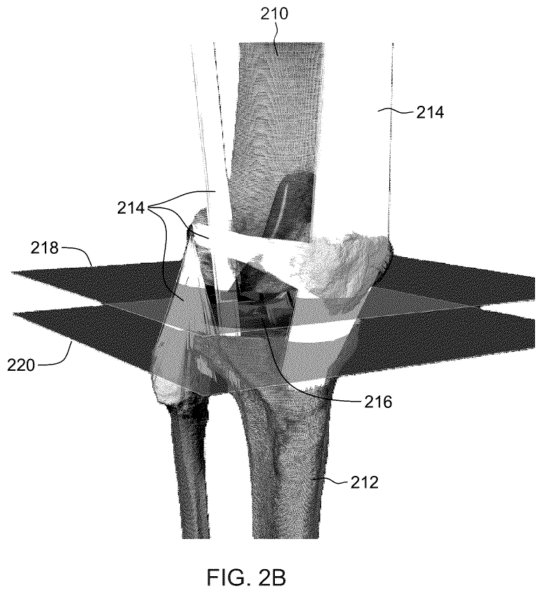

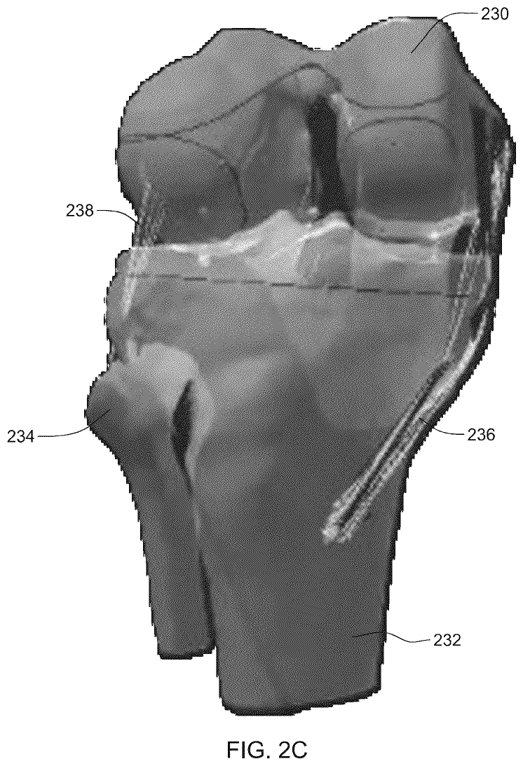

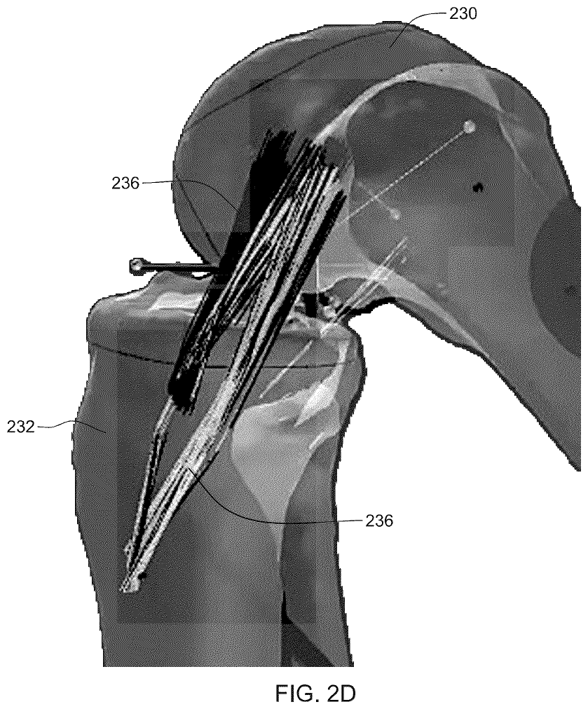

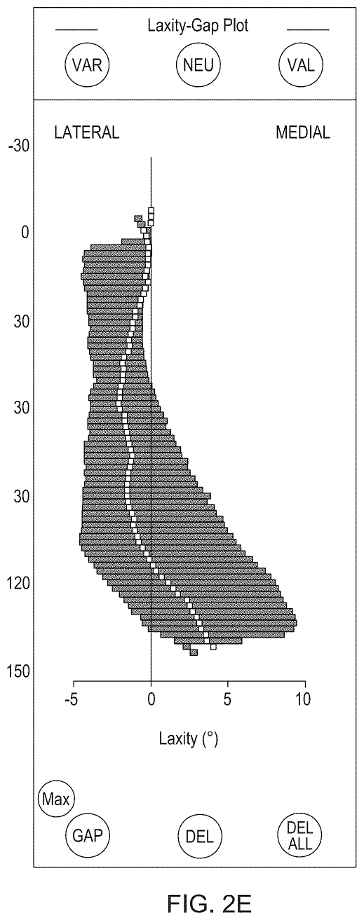

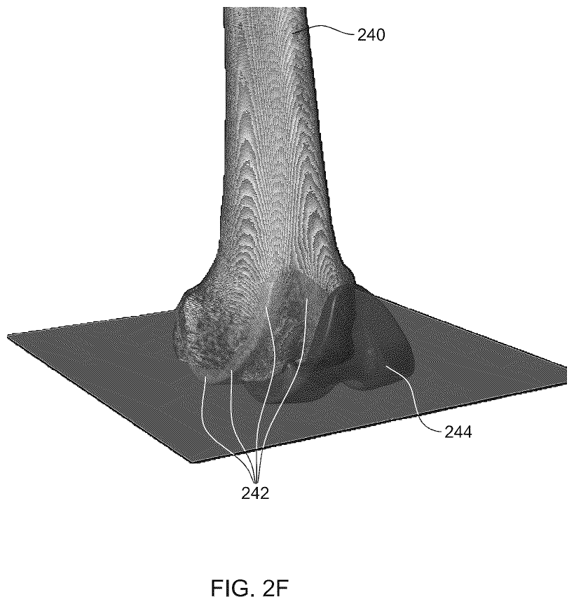

FIGS. 2A-2C help to illustrate various aspects described herein. Referring initially to FIG. 2A, FIG. 2A depicts an example of a visual CAD model and placement location for an implant superimposed to a tracked bone using augmented reality vision, in accordance with aspects described herein. In FIG. 2A, 202 is a digital model (e.g., point cloud, CAD model, etc.) of patient anatomy-bone in this example. It is presented in a heatmap style to reflect bone quality (for instance based on density); different colors/shades represent different quality/density. The brightest areas (presented in green for instance) are considered to reflect the best quality/densest bone. In other examples, color and/or brightness characteristics could be used to reflect relative stiffness or other characteristics of ligament/meniscus, cartilage, and muscle, as examples. 204 is a CAD model of an implant to be placed, and is shown in the desired/planned position for the implant relative to the bone being modeled. In examples, this positioning is constructed from information known prior to surgery, that is-patient anatomy may be known and a planned implant location may be known. The bone quality indication can be viewed by the surgeon in conjunction with the planned implant location for the surgeon to at least informally/subjectively gauge the desirability of the planned implant location. The surgeon could optionally move that location slightly, thus causing an update to the affected cut planes, etc. in the plan.

In examples in which a user views the surgical site through AR glasses, the implant model 204 could be shown in AR and the bone model could be shown as a graphical element in AR, if desired.

In one example, this is presented prior to cutting the bone. At that point, a next step may be initiated in which the cut planes are shown by graphical elements (e.g. colored planes). There are at least two cuts to be made in this example. The bone density model and/or implant graphical elements could optionally be modified, if desired. For instance, the implant model graphical element could be faded or removed so that the cut plane graphical elements are more easily viewed. The surgeon could execute the cuts optionally using a cut guide (which could be tracked if desired), or the cuts could be executed by way of robotic cutting, as examples. In any case, once the cuts have been made, then the bone model element(s) may be modified, for instance to display them in a neutral color and/or with relatively low opacity. Optionally the implant model could be displayed as such. In any event, the physical implant may be introduced to the field of view. This may be tracked. Optional other graphical element(s) could be displayed relative to that implant (for instance overlaid that implant if desired). As the implant is moved closer to the desired position as reflected by implant model 204, implant model 204 can change color as it is moved into the planned position for the implant. For instance, it can transition from red to green. In an example, the extent of the transition is a function of the proximity of the implant to the planned location. If the user were to hold the implant still and out of position, the element could remain static. As the user moves the implant closer, the color or other visual property could resume its transition.

FIG. 2B depicts an example of a visual CAD model of patient anatomy (a knee joint) showing femur 210 and tibia 212 bone graphical elements in a heatmap style with coloring/shading to represent bone density, as in FIG. 2A. Additionally depicted as elements 214 are ligaments of the knee joint. Also depicted as elements 216 are implants (e.g., a tibial tray and femoral component), as well two cut planes 218 and 220. This depiction may be provided on a standard display, though in some examples these graphical elements may be interposed in the line of sight of a user, e.g., in AR, as the user views the actual patient anatomy in the user's field of view during a surgical procedure.

FIGS. 2C-2D depict another example of a visual CAD model of patient anatomy (a knee joint) showing femur 230, tibia 232 and fibula 234 bone graphical elements, as well as ligaments of the knee joint presented with varying visual characteristics (e.g., color and/or brightness characteristics) to reflect the tension or slack those ligaments are under. Referring initially to FIG. 2C, the medial collateral ligament (MCL) 236 and lateral collateral ligament (LCL) 238 are shown in position relative to the bone anatomy and with varying color/shading at different portions of the respective ligaments to show corresponding tension/slack at those portions. FIG. 2D depicts a sagittal view of the knee joint, including the MCL 236 with varying color/shading at different portions of the MCL to show corresponding tension/slack at those portions. In examples, different colors could be used to represent different tensions. FIG. 2E depicts a laxity-gap plot corresponding to the ligament tensioning reflected in FIGS. 2C-2D. The plot presents lateral-medial laxity values in degrees (x-axis) at varying degrees of flexion (y-axis). Negative laxities correspond to a varus alignment and positive laxities corresponding to a valgus alignment. A system in accordance with aspects described herein can track tension in varus and valgus rotations to determine the soft tissue envelope. The information can be displayed granularly with the ligament tension characteristics (e.g., line color changes) so that users such as the surgeon know what is contributing to the soft tissue envelope graph at each flexion angle and determine to modify tension in a ligament, for instance to tension it more or to release tension to address an issue that the user sees.

FIG. 2F depicts an example visual CAD model of patient anatomy (a femur 240) graphical element in a heatmap style with coloring/shading to represent bone density. Also provided is a graphical element 242 (shown colored/shaded green for instance) showing intended placement of a femoral component implant, and another graphical element 244 (shown colored/shaded red for instance) showing actual location of the implant in real space as it is tracked in real time during the surgical procedure. In accordance with aspects described herein, the implant is introduced to the field of view and tracked. The system displays the graphical element 244, and places the element 244 to correspond to where the implant is currently in real space. As the surgeon moves the implant in real space closer to the desired position in real space, as reflected by graphical element 242, one or both of the graphical elements 242, 244 can change color/shade, which could be gradual or could be in full once the implant is entirely in the proper position in real space. For instance, graphical element 244 representing the current position of the physical implant during surgery can change from red to green, or the green graphical element could change from green to red. As yet another option, both elements 242, 244 could change to a different color, such as white, once the implant is properly placed. Any visual characteristics of one or both elements could be changed to indicate partial or full proper placement.

More generally, any sensory guidance can be used to alert a user as to the status of an object's position relative to a correct or intended location for that object, or areas of risk, such as proximity to nerve or arterial features. In addition or as an alternative to changing properties of a graphical element representing the object and/or intended position/location of the object, there could be sound or light that may be provided or removed depending on the position of the object. Other examples are possible.

In some embodiments, the tracking of patient anatomy to register the anatomy location (rather than relying solely on tracking an alignment guide or other object that fits to the anatomy) may be employed. Graphical elements for display to assist the surgery may be placed based on this anatomical tracking. For instance, graphical elements for cut planes and implant placement may be provided based on the anatomy tracking.

In some embodiments, aspects may be employed during remote surgery in which a user is not located in the OR but is instead in any arbitrary remote location. As long as the user has a view to the environment, the user can manipulate graphical elements to, for instance, guide a local user (in the OR) for placement or positioning of object or surgical actions like cutting and drilling. Additionally, the remote user could drive operation of a local robot (in the OR) to effect surgical actions to accomplish the surgical tasks.

In some embodiments, object usage may be tracked based on the system ‘seeing’ the object and its use during surgeries. For instance, the usage counts (number of times objects have been used) may be tracked for instruments. This can inform the need or desire for repair and/or replacement of these instruments based on meeting/exceeding thresholds, such as those based on manufacturer recommended exchange time or the like. In this manner, object wear and tear may be tracked, assessed, logged, gauged, etc., to trigger notifications, alerts, or the like for maintenance or replacement of the object.

Advantageously, aspects described herein can reduce surgical time, reduce infection and fracture risk using an alignment guide approach, and increase reproducibility compared to conventional planned surgery and execution thereof. Further, prior surgical methods rely on old navigation methodologies that require secondary registration by unqualified staff. Aspects described herein remove the time and training needed to interact with such technology. Aspects described herein can be used in any surgical application, as well as initial and rehabilitative patient visits to track objects relevant for the tasks being performed. Additionally, aspects can be used for surgical education, remote surgery, and remote viewing.

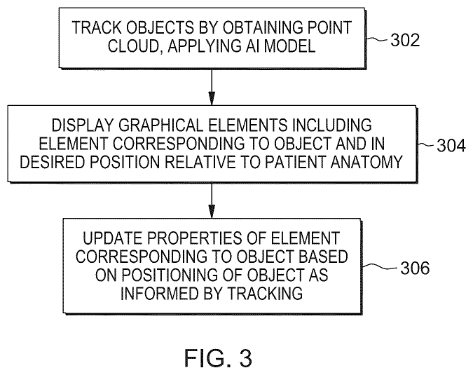

FIG. 3 depicts an example process for graphically guiding a surgical workflow of a surgical procedure, in accordance with aspects described herein. The process may be executed, in one or more examples, by a processor or processing circuitry of one or more computers/computer systems, such as those described herein. In one example, code or instructions implementing the process(es) of FIG. 3 are part of one or more code modules and/or one or more code sub-modules of the one or more modules. Various options are available.

The process of FIG. 3 includes tracking (302) one or more objects. This is done by obtaining point cloud data based on imaging one or more fields of view during the surgical procedure, applying an artificial intelligence model to the obtained point cloud data, and recognizing, based on the applying, (i) one or more objects in the one or more fields of view and (ii) positioning of the one or more objects in the one or more fields of view. Further, the one or more objects include an object to be placed as part of the surgical procedure.

The tracking (302) could track any of varying types of objects. In an embodiment, a tracked object includes an implant to be placed relative to the patient anatomy. Additionally or alternatively, a tracked object can include a surgical alignment guide. As an example, the surgical alignment guide is a cut guide. Additionally or alternatively, a tracked object can include a surgical tool used in the surgical procedure.

Continuing with FIG. 3, the process displays (304) graphical elements, for instance on a display device. The displayed graphical elements include, for example, at least one graphical element corresponding to the object and in a desired position relative to patient anatomy that is displayed as a live view to the patient anatomy and/or as a digital model of the patient anatomy on the display device. In some embodiments, the display device is a display device of smart glasses through which a user has a field of view of the one or more fields of view. Additionally or alternatively a display device could be a monitor/display screen that is viewable to users.

In embodiments of a tracked cut guide, the at least one graphical element can include at least one representation of the cut guide. The method in these instances, and specifically displaying the graphical elements, can further include displaying, on the display device, additional graphical elements that correspond to cut planes associated with cuts be made as facilitated by the cut guide.

Additionally or alternatively, the process can display, on the display device, an additional graphical element that corresponds to a drill hole location or screw placement location, where displaying the additional graphical element includes positioning the additional graphical element in a desired position for the drill hole or screen placement relative to the patient anatomy that is displayed as a live view and/or as a digital model on the display device.

In examples, the patient anatomy includes bone and/or soft tissue having varying densities or tension at different portions of the patient anatomy. The process can therefore display an additional graphical element representing the patient anatomy, where different portions of the additional graphical element correspond to the different portions of the patient anatomy, and where displaying the additional graphical element varies properties of the additional graphical element at the different portions of the additional graphical element according to the varying densities or tension at the corresponding different portions of the patient anatomy. An example of this is providing variations in shading and/or color of the anatomy at different locations to reflect density and/or tension at those different locations.

The process of FIG. 3. further includes updating (306) properties of the at least one graphical element, where the updating is based on detected positioning of the object in the one or more fields of view, as informed by the tracking. In examples, properties of graphical elements, such as the at least one graphical element, can include one or more colors or shading of the graphical element.

In some embodiments, the AI model is trained by an object calibration approach that can include pre-operatively imaging the object to be placed from varying angles and capturing a point-cloud of the object to be placed, and training the AI model using the point cloud to recognize the object to be placed during the surgical procedure.

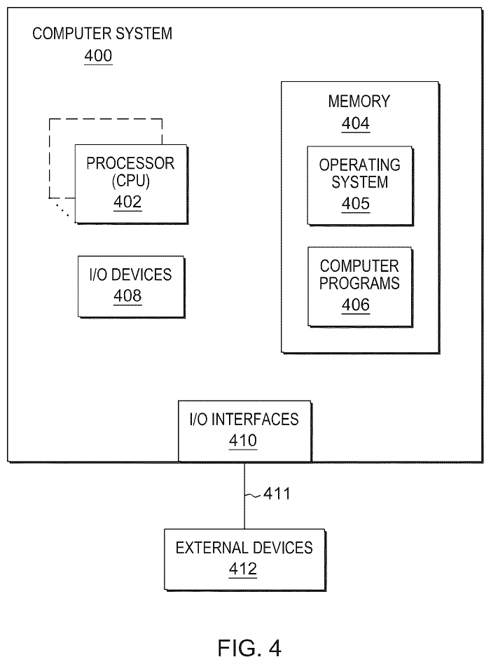

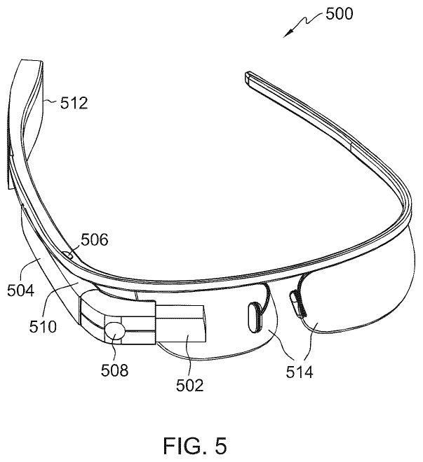

Processes described herein may be performed singly or collectively by one or more computer systems, such as one or more systems that are, or are in communication with an imaging, vision, and/or AR system or devices, as examples. FIG. 4 depicts one example of such a computer system and associated devices to incorporate and/or use aspects described herein. A computer system may also be referred to herein as a data processing device/system, computing device/system/node, or simply a computer. The computer system may be based on one or more of various system architectures and/or instruction set architectures, such as those offered by Intel Corporation (Santa Clara, California, USA) or ARM Holdings plc (Cambridge, England, United Kingdom), as examples.

FIG. 4 shows a computer system 400 in communication with external device(s) 412. Computer system 400 includes one or more processor(s) 402, for instance central processing unit(s) (CPUs). A processor can include functional components used in the execution of instructions, such as functional components to fetch program instructions from locations such as cache or main memory, decode program instructions, and execute program instructions, access memory for instruction execution, and write results of the executed instructions. A processor 402 can also include register(s) to be used by one or more of the functional components. Computer system 400 also includes memory 404, input/output (I/O) devices 403, and I/O interfaces 410, which may be coupled to processor(s) 402 and each other via one or more buses and/or other connections. Bus connections represent one or more of any of several types of bus structures, including a memory bus or memory controller, a peripheral bus, an accelerated graphics port, and a processor or local bus using any of a variety of bus architectures. By way of example, and not limitation, such architectures include the Industry Standard Architecture (ISA), the Micro Channel Architecture (MCA), the Enhanced ISA (EISA), the Video Electronics Standards Association (VESA) local bus, and the Peripheral Component Interconnect (PCI).

Memory 404 can be or include main or system memory (e.g., Random Access Memory) used in the execution of program instructions, storage device(s) such as hard drive(s), flash media, or optical media as examples, and/or cache memory, as examples. Memory 404 can include, for instance, a cache, such as a shared cache, which may be coupled to local caches (examples include L1 cache, L2 cache, etc.) of processor(s) 402. Additionally, memory 404 may be or include at least one computer program product having a set (e.g., at least one) of program modules, instructions, code or the like that is/are configured to carry out functions of embodiments described herein when executed by one or more processors.

Memory 404 can store an operating system 405 and other computer programs 406, such as one or more computer programs/applications that execute to perform aspects described herein. Specifically, programs/applications can include computer readable program instructions that may be configured to carry out functions of embodiments of aspects described herein.

Examples of I/O devices 408 include but are not limited to microphones, speakers, Global Positioning System (GPS) devices, RGB and/or IR cameras, lights, accelerometers, gyroscopes, magnetometers, sensor devices configured to sense light, proximity, heart rate, body and/or ambient temperature, blood pressure, and/or skin resistance, registration probes and activity monitors. An I/O device may be incorporated into the computer system as shown, though in some embodiments an I/O device may be regarded as an external device (412) coupled to the computer system through one or more I/O interfaces 410.

Computer system 400 may communicate with one or more external devices 412 via one or more I/O interfaces 410. Example external devices include a keyboard, a pointing device, a display, and/or any other devices that enable a user to interact with computer system 400. Other example external devices include any device that enables computer system 400 to communicate with one or more other computing systems or peripheral devices such as a printer. A network interface/adapter is an example I/O interface that enables computer system 400 to communicate with one or more networks, such as a local area network (LAN), a general wide area network (WAN), and/or a public network (e.g., the Internet), providing communication with other computing devices or systems, storage devices, or the like. Ethernet-based (such as Wi-Fi) interfaces and Bluetooth® adapters are just examples of the currently available types of network adapters used in computer systems (BLUETOOTH is a registered trademark of Bluetooth SIG, Inc., Kirkland, Washington, U.S.A.).

The communication between I/O interfaces 410 and external devices 412 can occur across wired and/or wireless communications link(s) 411, such as Ethernet-based wired or wireless connections. Example wireless connections include cellular, Wi-Fi, Bluetooth®, proximity-based, near-field, or other types of wireless connections. More generally, communications link(s) 411 may be any appropriate wireless and/or wired communication link(s) for communicating data.

Particular external device(s) 412 may include one or more data storage devices, which may store one or more programs, one or more computer readable program instructions, and/or data, etc. Computer system 400 may include and/or be coupled to and in communication with (e.g., as an external device of the computer system) removable/non-removable, volatile/non-volatile computer system storage media. For example, it may include and/or be coupled to a non-removable, non-volatile magnetic media (typically called a “hard drive”), a magnetic disk drive for reading from and writing to a removable, non-volatile magnetic disk (e.g., a “floppy disk”), and/or an optical disk drive for reading from or writing to a removable, non-volatile optical disk, such as a CD-ROM, DVD-ROM or other optical media.

Computer system 400 may be operational with numerous other general purpose or special purpose computing system environments or configurations. Computer system 400 may take any of various forms, well-known examples of which include, but are not limited to, personal computer (PC) system(s), server computer system(s), such as messaging server(s), thin client(s), thick client(s), workstation(s), laptop(s), handheld device(s), mobile device(s)/computer(s) such as smartphone(s), tablet(s), and wearable device(s), multiprocessor system(s), microprocessor-based system(s), telephony device(s), network appliance(s) (such as edge appliance(s)), virtualization device(s), storage controller(s), set top box(es), programmable consumer electronic(s), network PC(s), minicomputer system(s), mainframe computer system(s), and distributed cloud computing environment(s) that include any of the above systems or devices, and the like.

FIG. 4 depicts another example of a computer system to incorporate and use aspects described herein. FIG. 4 depicts an example eyewear based wearable device, for instance a wearable smart glasses device to facilitate presentation of AR elements to a wearer of the device. Device 400 can include many of the same types of components included in computer system 400 described above. In the example of FIG. 4, device 400 is configured to be wearable on the head of the device user. The device includes a display 402 that is positioned in a peripheral vision line of sight of the user when the device is in operative position on the user's head. Suitable displays can utilize LCD, CRT, or OLED display technologies, as examples. Lenses 414 may optionally include active translucent displays, in which an inner and/or outer surface of the lenses are capable of displaying images and other content. This provides the ability to impose this content directly into the line of sight of the user, overlaying at least part of the user's view to the environment through the lenses. In particular embodiments described herein, graphical elements presented on the lens displays are AR elements overlaying a view to the environment.

Device 400 also includes touch input portion 404 that enable users to input touch-gestures in order to control functions of the device. Such gestures can be interpreted as commands, for instance a command to take a picture, or a command to launch a particular service. Device 400 also includes button 406 in order to control function(s) of the device. Example functions include locking, shutting down, or placing the device into a standby or sleep mode.

Various other input devices are provided, such as camera 408, which can be used to capture images or video. The camera can be used by the device to obtain image(s)/video of a view of the wearer's environment to use in, for instance, capturing images/videos of a scene. Additionally, camera(s) may be used to track the user's direction of eyesight and ascertain where the user is looking, and track the user's other eye activity, such as blinking or movement.

One or more microphones, proximity sensors, light sensors, accelerometers, speakers, GPS devices, and/or other input devices (not labeled) may be additionally provided, for instance within housing 410. Housing 410 can also include other electronic components, such as electronic circuitry, including processor(s), memory, and/or communications devices, such as cellular, short-range wireless (e.g., Bluetooth), or Wi-Fi circuitry for connection to remote devices. Housing 410 can further include a power source, such as a battery to power components of device 400. Additionally or alternatively, any such circuitry or battery can be included in enlarged end 412, which may be enlarged to accommodate such components. Enlarged end 412, or any other portion of device 400, can also include physical port(s) (not pictured) used to connect device 400 to a power source (to recharge a battery) and/or any other external device, such as a computer. Such physical ports can be of any standardized or proprietary type, such as Universal Serial Bus (USB).

Aspects of the present invention may be a system, a method, and/or a computer program product, any of which may be configured to perform or facilitate aspects described herein. Computer system configured to perform these and other methods, and computer program products that include a computer readable storage medium storing instructions for execution to perform these and other methods are also provided.

In some embodiments, aspects of the present invention may take the form of a computer program product, which may be embodied as computer readable medium(s). A computer readable medium may be a tangible storage device/medium having computer readable program code/instructions stored thereon. Example computer readable medium(s) include, but are not limited to, electronic, magnetic, optical, or semiconductor storage devices or systems, or any combination of the foregoing. Example embodiments of a computer readable medium include a hard drive or other mass-storage device, an electrical connection having wires, random access memory (RAM), read-only memory (ROM), erasable-programmable read-only memory such as EPROM or flash memory, an optical fiber, a portable computer disk/diskette, such as a compact disc read-only memory (CD-ROM) or Digital Versatile Disc (DVD), an optical storage device, a magnetic storage device, or any combination of the foregoing. The computer readable medium may be readable by a processor, processing unit, or the like, to obtain data (e.g., instructions) from the medium for execution. In a particular example, a computer program product is or includes one or more computer readable media that includes/stores computer readable program code to provide and facilitate one or more aspects described herein.

As noted, program instruction contained or stored in/on a computer readable medium can be obtained and executed by any of various suitable components such as a processor of a computer system to cause the computer system to behave and function in a particular manner. Such program instructions for carrying out operations to perform, achieve, or facilitate aspects described herein may be written in, or compiled from code written in, any desired programming language. In some embodiments, such programming language includes object-oriented and/or procedural programming languages such as C, C++, C#, Java, etc.

Program code can include one or more program instructions obtained for execution by one or more processors. Computer program instructions may be provided to one or more processors of, e.g., one or more computer systems, to produce a machine, such that the program instructions, when executed by the one or more processors, perform, achieve, or facilitate aspects of the present invention, such as actions or functions described in flowcharts and/or block diagrams described herein. Thus, each block, or combinations of blocks, of the flowchart illustrations and/or block diagrams depicted and described herein can be implemented, in some embodiments, by computer program instructions.

Although various embodiments are described above, these are only examples.

The terminology used herein is for the purpose of describing particular embodiments only and is not intended to be limiting. As used herein, the singular forms “a”, “an” and “the” are intended to include the plural forms as well, unless the context clearly indicates otherwise. It will be further understood that the terms “comprises” and/or “comprising”, when used in this specification, specify the presence of stated features, integers, steps, operations, elements, and/or components, but do not preclude the presence or addition of one or more other features, integers, steps, operations, elements, components and/or groups thereof.

The corresponding structures, materials, acts, and equivalents of all means or step plus function elements in the claims below, if any, are intended to include any structure, material, or act for performing the function in combination with other claimed elements as specifically claimed. The description of one or more embodiments has been presented for purposes of illustration and description, but is not intended to be exhaustive or limited to in the form disclosed. Many modifications and variations will be apparent to those of ordinary skill in the art. The embodiment was chosen and described in order to best explain various aspects and the practical application, and to enable others of ordinary skill in the art to understand various embodiments with various modifications as are suited to the particular use contemplated.

What is claimed is:

1. A computer-implemented method including:
graphically guiding a surgical workflow of a surgical procedure, the graphically guiding including:
tracking one or more objects by:
obtaining point cloud data based on imaging one or more fields of view during the surgical procedure; and
applying an artificial intelligence model to the obtained point cloud data, and recognizing, based on the applying, one or more objects in the one or more fields of view, and positioning of the one or more objects in the one or more fields of view, the one or more objects including an object to be placed as part of the surgical procedure;
displaying at least one graphical element corresponding to the object on a display device and in a desired position relative to patient anatomy that is displayed as a live view to the patient anatomy and/or as a digital model of the patient anatomy on the display device; and
updating properties of the at least one graphical element, wherein the updating is based on detected positioning of the object in the one or more fields of view, as informed by the tracking.

2. The method of claim 1, wherein the object includes an implant to be placed relative to the patient anatomy.

3. The method of claim 1, wherein the object includes a surgical alignment guide.

4. The method of claim 3, wherein the surgical alignment guide is a cut guide, wherein the at least one graphical element includes at least one representation of the cut guide, and wherein the method further includes displaying, on the display device, additional graphical elements that correspond to cut planes associated with cuts be made.

5. The method of claim 1, wherein the object includes a surgical tool used in the surgical procedure.

6. The method of claim 1, further including displaying, on the display device, an additional graphical element that corresponds to a drill hole location or screw placement location, wherein the displaying the additional graphical element includes positioning the additional graphical element in a desired position for the drill hole or screen placement relative to the patient anatomy that is displayed as a live view and/or as a digital model on the display device.

7. The method of claim 1, wherein the display device is a display device of smart glasses through which a user has a field of view of the one or more fields of view.

8. The method of claim 1, wherein the properties of the at least one graphical element include one or more colors or shading of the at least one graphical element.

9. The method of claim 1, wherein the patient anatomy includes bone and/or soft tissue having varying densities or tension at different portions of the patient anatomy, and wherein the method further includes displaying an additional graphical element representing the patient anatomy, wherein different portions of the additional graphical element correspond to the different portions of the patient anatomy, and wherein displaying the additional graphical element varies properties of the additional graphical element at the different portions of the additional graphical element according to the varying densities or tension at the corresponding different portions of the patient anatomy.

10. The method of claim 1, further including training the AI model by an object calibration approach that includes:
pre-operatively imaging the object to be placed from varying angles and capturing a point-cloud of the object to be placed; and
training the AI model using the point cloud to recognize the object to be placed during the surgical procedure.

11. A computer system including:
a memory; and
a processing circuit in communication with the memory, wherein the computer system is configured to perform:
graphically guiding a surgical workflow of a surgical procedure, the graphically guiding including:
tracking one or more objects by:
obtaining point cloud data based on imaging one or more fields of view during the surgical procedure; and
applying an artificial intelligence model to the obtained point cloud data, and recognizing, based on the applying, one or more objects in the one or more fields of view, and positioning of the one or more objects in the one or more fields of view, the one or more objects including an object to be placed as part of the surgical procedure;
displaying at least one graphical element corresponding to the object on a display device and in a desired position relative to patient anatomy that is displayed as a live view to the patient anatomy and/or as a digital model of the patient anatomy on the display device; and
updating properties of the at least one graphical element, wherein the updating is based on detected positioning of the object in the one or more fields of view, as informed by the tracking.

12. The computer system of claim 11, wherein the object includes:
an implant to be placed relative to the patient anatomy; or
a surgical tool used in the surgical procedure.

13. The computer system of claim 11, wherein the object includes a surgical alignment guide, wherein the surgical alignment guide is a cut guide, wherein the at least one graphical element includes at least one representation of the cut guide, and wherein the computer system is further configured to perform displaying, on the display device, additional graphical elements that correspond to cut planes associated with cuts be made.

14. The computer system of claim 11, wherein the patient anatomy includes bone and/or soft tissue having varying densities or tension at different portions of the patient anatomy, and wherein the computer system is further configured to perform displaying an additional graphical element representing the patient anatomy, wherein different portions of the additional graphical element correspond to the different portions of the patient anatomy, and wherein displaying the additional graphical element varies properties of the additional graphical element at the different portions of the additional graphical element according to the varying densities or tension at the corresponding different portions of the patient anatomy.

15. The computer system of claim 11, wherein the computer system is further configured to perform training the AI model by an object calibration approach that includes:
pre-operatively imaging the object to be placed from varying angles and capturing a point-cloud of the object to be placed; and
training the AI model using the point cloud to recognize the object to be placed during the surgical procedure.

16. A computer program product including:
a computer readable storage medium readable by a processing circuit and storing instructions for execution by the processing circuit to perform:
graphically guiding a surgical workflow of a surgical procedure, the graphically guiding including:
tracking one or more objects by:
obtaining point cloud data based on imaging one or more fields of view during the surgical procedure; and
applying an artificial intelligence model to the obtained point cloud data, and recognizing, based on the applying, one or more objects in the one or more fields of view, and positioning of the one or more objects in the one or more fields of view, the one or more objects including an object to be placed as part of the surgical procedure;
displaying at least one graphical element corresponding to the object on a display device and in a desired position relative to patient anatomy that is displayed as a live view to the patient anatomy and/or as a digital model of the patient anatomy on the display device; and
updating properties of the at least one graphical element, wherein the updating is based on detected positioning of the object in the one or more fields of view, as informed by the tracking.

17. The computer program product of claim 16, wherein the object includes:
an implant to be placed relative to the patient anatomy; or
a surgical tool used in the surgical procedure.

18. The computer program product of claim 16, wherein the object includes a surgical alignment guide, wherein the surgical alignment guide is a cut guide, wherein the at least one graphical element includes at least one representation of the cut guide, and wherein the instructions for execution by the processing circuit are further to perform displaying, on the display device, additional graphical elements that correspond to cut planes associated with cuts be made.

19. The computer program product of claim 16, wherein the patient anatomy includes bone and/or soft tissue having varying densities or tension at different portions of the patient anatomy, and wherein the instructions for execution by the processing circuit are further to perform displaying an additional graphical element representing the patient anatomy, wherein different portions of the additional graphical element correspond to the different portions of the patient anatomy, and wherein displaying the additional graphical element varies properties of the additional graphical element at the different portions of the additional graphical element according to the varying densities or tension at the corresponding different portions of the patient anatomy.

20. The computer program product of claim 16, wherein the instructions for execution by the processing circuit are further to perform training the AI model by an object calibration approach that includes:
pre-operatively imaging the object to be placed from varying angles and capturing a point-cloud of the object to be placed; and
training the AI model using the point cloud to recognize the object to be placed during the surgical procedure.
